(19) United States
(12) Patent Application Publication
(10) Pub. No.: US 2022/0378959 A1
(43) Pub. Date: Dec. 1, 2022

(54) A SYSTEM FOR TREATING A SURFACE COMPRISING AN ULTRAVIOLET LIGHTING ARRANGEMENT

(71) Applicant: LIGHTLAB SWEDEN AB, Uppsala (SE)

(72) Inventor: Jonas TIRÉN, Uppsala (SE)

(73) Assignee: LIGHTLAB SWEDEN AB, Uppsala (SE)

(21) Appl. No.: 17/776,521

(22) PCT Filed: Nov. 16, 2020
(86) PCT No.: PCT/SE2020/051095

§ 371 Date: May 12, 2022

(30) Foreign Application Priority Data
Nov. 18, 2019 (SE) 1951325-8

(51) Int. Cl. A61L 2/10

(52) U.S. Cl. CPC A61L 2/10; A61L 2202/11; A61L 2202/14

(57) ABSTRACT

The present disclosure generally relates to a system for treating a surface, comprising an ultraviolet (UV) lighting arrangement configured to emit UV light towards the surface at a first and a second wavelength range to effectively reduce microorganisms at the surface.

### TECHNICAL FIELD

The present disclosure generally relates to a system for treating a surface, comprising an ultraviolet (UV) lighting arrangement configured to emit UV light towards the surface at a first and a second wavelength range to effectively reduce microorganisms at the surface.

### BACKGROUND

Systems for disinfection of water, air, surfaces or certain equipment using ultraviolet (UV) light generated by low pressure mercury lamps (LP-Hg lamps), predominantly emitting wavelengths around 254 nm, are commonly used today. Medium and/or high-pressure Hg lamps may alternatively be used, for example in large systems such as for water disinfection as these lamps may deliver higher power output. These systems may be combined with particle filtering, reverse osmosis (for water disinfection) and other. The UVC-systems are popular since they do not use any chemicals (e.g. chlorine), which is advantageous for many reasons, environmental not the least.

These light sources work well, can have a good energy efficiency (for larger low-pressure Hg lamps this may in the range of 30-35%) and have lifetimes that for the best products today, are well above 10 000 hours; 16 000 hours is reported for the best products. Other UV sources (e.g. Excimer light sources) exist but have reportedly either a very short lifetime (<500 hours) or a very low energy efficiency (e.g. UVC-LED typically in the order of 1-2%).

A serious drawback with LP-Hg light sources is that the light source de-activates (kills) bacteria to a certain level, after which no significant reduction is seen. This phenomenon is generally referred to as “tailing”. There are several (somewhat different) explanations found in the literature for this; the most accepted is that the bacteria will have a process of self-repair (also called re-activation, and auto-repair). If the rate of such a self-repair process is the same as the de-activation process the net result would be a steady state condition. Typically, in tests performed, Hg-lamps do not reach below 10²-10³ Colony Forming Units per milliliter (CFU/ml) for a generally used microorganism for this kind of testing and validation, i.e. Escherichia coli bacteria (E. coli). Now, after the disinfection the still active remaining micro-organisms will start to multiply again, after a so-called lag period, i.e. an initial period under which no growth is observed. For E. coli the doubling rate, a.k.a. the generation rate, i.e. the time it takes for the bacteria to double their numbers, may typically be considered to be in the order of 20 minutes at room temperature. This rate is depending on many other parameters such as temperature, pH, access to nutrient etc. In water that has been disinfected, the nutrients may for example be the de-activated microorganisms. An unwanted consequence of this is that the disinfected water typically will become re-infected after some period of time.

A solution trying to contravene this problem is disclosed in US20190298879, including a dual light source solution where light emitted by a mercury-based UV light source is combined with light emitted by a non-mercury field emission-based UV light source. The solution in US20190298879 is specifically targeted towards treating a fluid in a container, where the fluid is allowed to encircle the light sources.

Even though US20190298879 allows for a reduction of microorganisms in a fluid, such as when the fluid is encircling the two light sources, there is always a desire to introduce further improvements, with an overall desire to minimize microorganisms in areas that could impact the wellbeing of e.g. humans.

### SUMMARY

According to an aspect of the present disclosure, the above is at least partly alleviated by a system for treating a surface, comprising a UV lighting arrangement configured to emit UV light towards the surface, processing circuitry configured to control the operation of the UV lighting arrangement, wherein the UV lighting arrangement under the control of the processing circuitry is adapted to emit UV light within both a first and a second wavelength range to effectively reduce microorganisms at the surface, the first wavelength range has an upper limit extending up to at least 270 nm, and the second wavelength range has a lower limit extending down to at least 270 nm and an upper limit range extending to at least 320 nm.

As stated above, in accordance to the present disclosure there is provided a solution where the UV lighting arrangement is adapted to emit light within two possibly (e.g. slightly) overlapping wavelength ranges, defined as a first and a second wavelength range, where the first and the second wavelength range has a common end point in a vicinity of 270 nm. As such, the first wavelength range has a lower end point well below 270 nm and the second wavelength range has an upper end point well above 270 nm, specifically having an upper limit range extending to at least 320 nm. In possible embodiments the first wavelength range extends at least down to at least 250 nm.

In a general prior art solution involving UV light for treating a surface, the focus has been on solely applying “UV-C wavelengths” (specifically at 254 nm), traditionally referred to as germicidal UV. However, by means of the present disclosure UV light being also within the UV-A (320-400 nm) and UV-B (280-320 nm) wavelength ranges are used for treating the surface. The present inventor has specifically identified that UV light at around 320 nm have great effectiveness against certain bacteria, such as for example the above mentioned E. coli bacteria.

Furthermore, by applying the UV light within more than one wavelength range as is achieved by means of the UV lighting arrangement (emitting UV light within both the first and the second wavelength range) is has shown to be possible to greatly reduce photo-reactivation, the process that can result in self-repair of damaged microbes. In principle it is known that a self-repair process (also called re-activation) may be occurring. This process involves proteins that are damaged by higher wavelengths than the de-activation process. Another theory for the tailing is that a small portion of the micro-organisms are more resistant to UV radiation, thus needing a much larger UV dose to be de-activated. However, from the measurements performed this seems not to be a large effect at least for the tested micro-organism, e-coli ATCC 8739.

By means of the present disclosure, the tailing effect may also be reduced in relation to surfaces, such as for example in relation to surfaces that may come in direct or indirect contact with a person. The overall advantage following with the present disclosure is thus that the risk of a decease involving the human/person is greatly reduced.

Within the context of the present disclosure it should be understood that the UV lighting arrangement must not necessarily emit light throughout all of the first and the wavelength range. Rather, it may sufficiently to be within the scope of the present disclosure to ensure that at least UV light with a narrow range and thus having a peak wavelength is emitted within each of the wavelength ranges. In one possible embodiment it may for example be possible to arrange the UV lighting arrangement to emit light having a peak wavelength at 260 nm (i.e. within the first wavelength range) and light having a peak wavelength at 300 nm (i.e. within the second wavelength range). Possibly, the peak wavelength is in such an embodiment distinct, meaning that an intensity at the peak wavelength is at least 50%-70% higher than at wavelengths “surrounding” the peak wavelength.

It could also be possible to arrange the UV lighting arrangement to emit light having a broad emission within e.g. the first wavelength range and emitting light having a distinct narrow banded peak wavelength within the second wavelength range. The opposite is of course possible, with narrow banded emission within the first wavelength range and an in comparison broader emission within the second wavelength range. In a preferred embodiment, the UV lighting arrangement comprises a first UV light source adapted to emit UV light within the first wavelength range and a second UV light source adapted to emit UV light within the second wavelength range. Possibly, the first light source may be configured to emit radiation within a wavelength range from around 240 nm to at least 270 nm and the second UV light source possibly configured to emit radiation within a wavelength range from around 270 nm to at least 320 nm. It is preferred to allow the first UV light source to emit light having a wavelength interval including emission of UV radiation at 265 nm, being a possible peak value for germicidal effectiveness. In another preferred embodiment, a single light source is adapted to emit UV radiation in a broader spectrum, i.e. covering both wavelength ranges using a single device.

In a preferred embodiment of the present disclosure, the first UV light source comprises e.g. a plurality of UV(C)-LEDs and/or a combination of light sources based on different technologies to suit the application. The UVC-LEDs may additionally have several different wavelength peaks in order to better cover a specific wavelength range.

Furthermore, emerging technologies such as field emission light sources (FEL) may be used in relation to the present disclosure and offers turn on times that are in the order of milliseconds, mainly governed by the electronic drive unit. In comparison, Hg-LP lamps typically need a warmup time in the range of a few minutes before they will reach full output power. UVC-LEDs are currently being developed but are at this time exhibiting reportedly short lifetimes and very low energy efficiencies. Significant efforts are being used in order to improve this and may surely and eventually be successful. Field emission light sources on the other hand may have lifetimes in the order of 1000-10000 hours depending on the desired power density and have been measured to reach efficiencies around 10%, albeit 4-5% in the UVC region.

An advantageous effect with using a field emission light source as the first UV light source is that such a light source may be configured to emit UV light at a spectrum that is not a distinct peak around 254 nm but a more continuous spectrum in above the mentioned range of 240-320 nm. Field emission UVC lamps have demonstrated the capability to continue the disinfection process and do not exhibit any significant tailing effect.

The field emission light source may in one embodiment comprise a field emission cathode and an electrically conductive anode structure. The field emission cathode typically comprises a plurality of nanostructures formed on a substrate, whereas the electrically conductive anode structure comprises a light converting material arranged to receive electrons from the cathode and to emit UV light. The light converting material may for example be selected to be at least one of LaPO4:Pr³⁺, LuPO3:Pr³⁺, Lu2Si2O7:Pr³⁺, YBO3:Pr³⁺ or YPO4:Bi³⁺ or a similar light converting material (there may be other materials as well). As an alternative, the light converting material may generally be seen as a phosphor material.

Preferably, the nanostructures preferably comprise at least one of ZnO nanostructures and carbon nanotubes. The plurality of ZnO nanostructures is adapted to have a length of at least 1 μm. In another embodiment the nanostructures may advantageously have a length in the range of 3-50 μm and a diameter in the range of 5-300 nm.

Preferably, the field emission light source is provided with a UV light permeable portion comprises at least one of Quartz, fused silica, UV transparent borosilicate and UV transparent soft glass. Such materials are suitable due to their inherent transparency to UV light.

Generally, a material/structure is considered to be “transparent” to ultraviolet light of a particular wavelength when the material/structure allows a significant amount of the ultraviolet radiation to pass there through. In an embodiment, the ultraviolet transparent structure is formed of a material and has a thickness, which allows at least ten percent of the ultraviolet radiation to pass there through.

During operation of the field emission light source, an in comparison high voltage is applied between the cathode and the anode. The electron energy used for consumer applications should be less than 10 kV and preferably less than 9 kV or soft X-rays generated by Bremsstrahlung will be able to escape the light source (it is otherwise absorbed by the anode glass). However, these levels are to some extent depending on glass thickness, thus higher voltages can be allowed if a thicker glass is used.

On the other hand. the electron energy must be high enough to effectively generate UV radiation. A preferred range for consumer applications is thus 4-9 kV and 7-15 kV for industrial applications (where some soft X-rays may be accepted).

The cathode and the anode are in in one embodiment arranged in an evacuated chamber, where the evacuated chamber is arranged under partial vacuum so that the electrons emitted from the cathode may transit to the anode with only a small number of collisions with gas molecules. Frequently the evacuated space may be evacuated to a pressure of less than 1×10⁻⁴ Torr.

As mentioned above, the system comprises a processing circuitry configured to control the operation of the UV lighting arrangement. The processing circuitry may in some embodiments be adapted to operate of the non-mercury based UV light source according to a predefined schedule, where the predefined schedule for example may be dependent on at least one of a distance to the surface, a target micro-organism, or an expected user behavior. Further schedules are possible and within the scope of the present disclosure. The operation of the system based on the predefined schedule will be further elaborated below in relation to the detailed description.

It may further be desirable to arrange the system to further comprise a reflective portion, where the reflective portion is adapted to increase the amount of UV light that is intended to be used for minimizing microorganisms in areas that could impact the wellbeing of e.g. humans. As such, rather than “escaping” the UV light is as such focused towards the surface that is to be treated.

As used herein, a material/structure is considered to be “reflective” to ultraviolet light of a particular wavelength when the material/structure has an ultraviolet reflection coefficient of at least thirty percent for the ultraviolet light of the particular wavelength. In a more particular embodiment, a highly ultraviolet reflective material/structure has an ultraviolet reflection coefficient of at least eighty percent.

Preferably, the system according to the present disclosure may be arranged as a component of a larger arrangement, such as a refrigerator (or freezer), an air purifier, a HVAC unit, or a disinfection cabinet. In some applications typically an air filter is present and the filter itself is subjected to UVC-radiation. Exemplary implementations in line with the present disclosure will be further elaborated below.

Further features of, and advantages with, the present disclosure will become apparent when studying the appended claims and the following description. The skilled addressee realize that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIGS. 1A and 1B illustrate different embodiments a system for treating a surface, according to currently preferred embodiments of the present disclosure,

FIGS. 2A-2B shows exemplary implementations comprising the system as shown in FIG. 1,

FIGS. 3A-3B illustrates the emission spectra from an Hg light source and its corresponding germicidal de-activation curve,

FIGS. 4A-4F illustrates different emission spectra resulting from different phosphor material and their corresponding germicidal de-activation curves,

FIG. 5 illustrates emission spectrums for possible implementation of the system, and

FIG. 6 illustrates the results from a mathematical model with and without a re-activation process.

### DETAILED DESCRIPTION

The present disclosure will now be described more fully hereinafter with reference to the accompanying drawings, in which currently preferred embodiments of the present disclosure are shown. This present disclosure may, however, be embodied in many different forms and should not be construed as limited to the embodiments set forth herein; rather, these embodiments are provided for thoroughness and completeness, and fully convey the scope of the present disclosure to the skilled addressee. Like reference characters refer to like elements throughout.

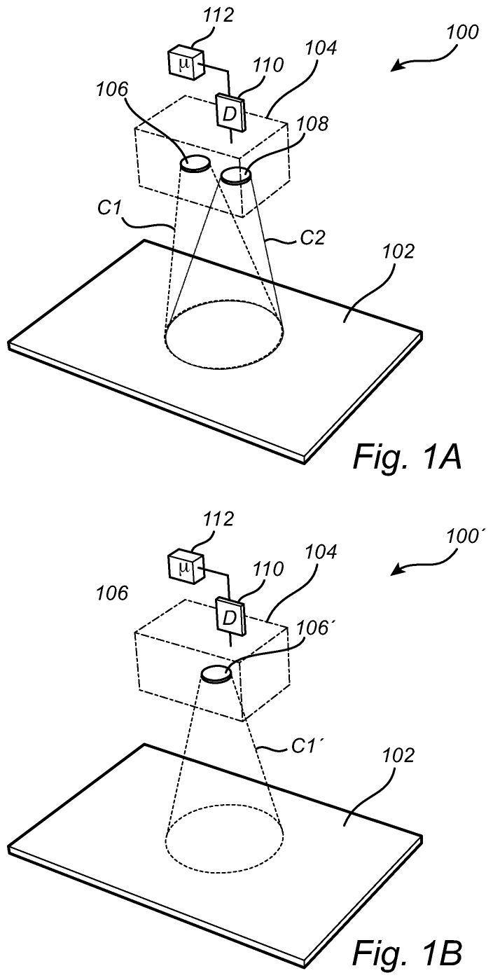

Referring now to the drawings and to FIG. 1A in particular, there is illustrated an embodiment of a system 100 for treating a surface 102. The system 100 comprises UV lighting arrangement 104 configured to emit UV light towards the surface 102. The UV lighting arrangement 104 in turn comprises a first 106 and a second 108 light source. The first light source 106 is adapted to emit UV light within a first wavelength range, where the first wavelength range has an upper limit extending to at least 270 nm. Also, the second UV light source 108 is adapted to emit UV light, however the second UV light 108 is arranged to emit light within a second wavelength range, where the second wavelength range has a lower limit extending to at least 270 nm.

In a preferred embodiment, the first 106 and the second 108 light source have a combine wavelength range extending between at least 250 nm-320 nm.

The system 100 further comprises a driver 110 connected to the UV lighting arrangement 104 and arranged to provide power for driving the light sources of the UV lighting arrangement 104. The system further comprises processing circuitry 112, arranged in communication with the driver 110 and arranged to control the overall operation of the driver 110 for controlling the light sources of the UV lighting arrangement 104. The processing circuitry 112 and the driver 110 may be integrated into a single unit.

Each of the first 106 and the second light source 108 are arranged to emit UV light (radiation) with an intensity distribution 114 as exemplified in FIG. 1A. As is illustrated, the first 106 and the second light source 108 are arranged such that they each emit light in a “cone” C1, C2, respectively, towards the surface 102. A typical cone angle may for example be selected to be between 45 degrees and 60 degrees.

Furthermore, in FIG. 1A the first 106 and the second 108 light source are arranged such that the cones C1, C2 essentially overlap. The area at the surface 102 where the cones overlap will accordingly be adapted to receive light within both the first and the second wavelength range.

As is readily understood, it may become necessary to allow the UV lighting arrangement 104 to comprise a plurality of light sources in order to cover a full area of the surface 102 with UV radiation.

It should also be noted that certain microorganism may be required to receive a specific dose UVC irradiation in order to deactivate the microorganisms on the surface to a specified level.

This dose D may be expressed as:

D=I×t

where I is the intensity, e.g. expressed in mW/cm² from the light sources (e.g. 106/108) onto the surface, e.g. surface 102, and t is the time during which the irradiation is applied.

The intensity I on the surface may then possibly be expressed as:

I=I(o)/A

where I(o) is the intensity as zero distance from the light sources and A is the area on surface 102 on which the radiation is to be distributed.
This area may in turn be expressed as:

π×r²

where r is the radius and is expressed as r=d×tan (v), where d is the distance between the surface and the UV source, and v is the cone angle as defined above.

In this slightly simplified example, the intensity is assumed constant over the beam angle and absorption in the media between the UV light sources and the surface 102 are neglected. More accurate calculations are entirely feasible and straight forward but are not deemed necessary in this description.

Now it is easy to realize that an increased distance may yield a larger area covered onto surface 102 by a single UV light source (e.g. each of the first 106 and the second 108 light source). This will however also result in a lower intensity I on this surface and the time to reach the required does D will be correspondingly increased.

Using two separate UV light sources 106, 108, each covering a specific wavelength range as described above, i.e. two different wavelength ranges in order to achieve both a high level of deactivation as well as preventing re-activation and tailing it may be advantageous to ensure that the areas subjected to irradiation from each of the two UV sources coincide, as presented in FIG. 1A. This may for example be achieved by tilting the two adjacent UV sources slightly in relation to each other, i.e. such that an angle of the each of the light sources 106, 108 facing the surface is arranged to be slightly different from each other. In one embodiment, the angle is selected dependent on the distance between the surface 102 and each of the light sources 106, 108.

In an alternative embodiment, as shown in FIG. 1B, the lighting arrangement 104 comprised with the system 100′ includes an alternative UV light source 106′, where the alternative UV light source 106′ by itself is adapted to have a wavelength range extending between at least 250 nm-320 nm. In such an embodiment, i.e. as shown in FIG. 1B, there is no need to consider overlapping cones as is shown in FIG. 1A. Rather, alternative UV light source 106′ presenting an alternative cone C1′ will provide both the wavelength ranges as discussed in relation to FIG. 1A.

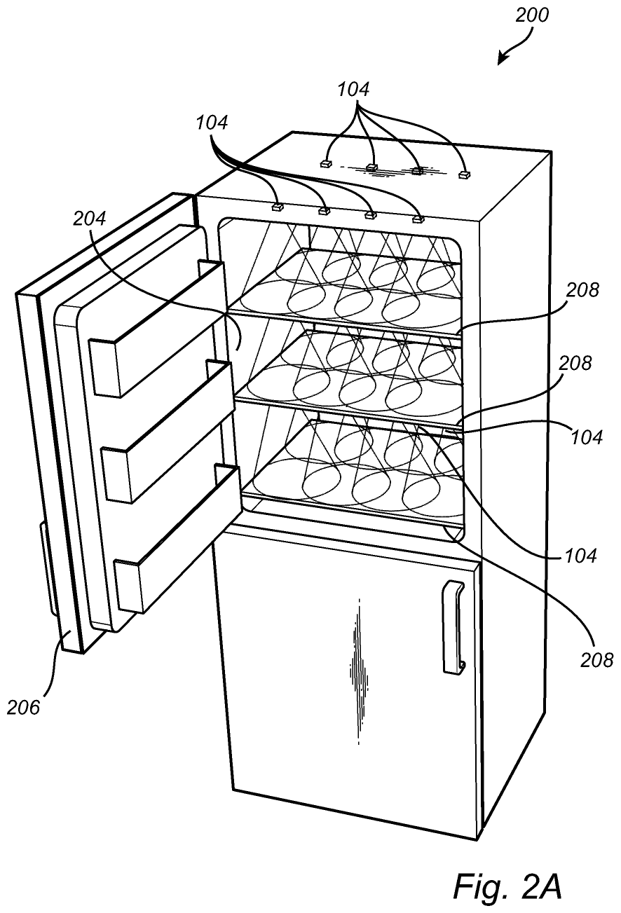

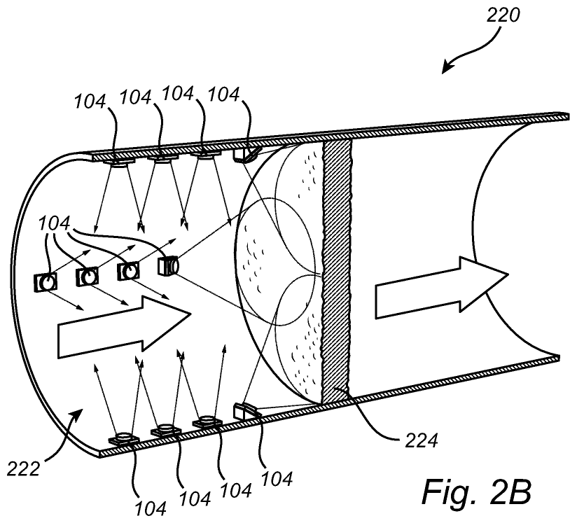

FIG. 2A shows a first exemplary embodiment where either of the systems 100 or 100′ may be included. Specifically, FIG. 2A shows a simplified view of fridge 202 according to embodiments of the present disclosure. A sealable compartment 204 includes a door 206 disposed on at least one side to allow access to compartment 204. A refrigerating circuit (not explicitly shown) may for example be used for cooling an interior of compartment 204.

The compartment 204 may be insulated. Any or all of the interior walls of compartment 204, including top, bottom, and side walls, may be UV-reflective material or coated with a UV-reflective material, in order to further distribute the UV energy. The compartment 204, door 206, the seal between door 206 and compartment 204, etc. may be provided by means of any form of suitable components as previously known to the skilled person.

In line with the present disclosure, the system 100/100′ may be such arranged that a plurality of light sources comprised with the system 100/100′ emit UV light within the compartment 204. As is exemplified in FIG. 2A, six UV lighting arrangements 104 are installed such that they emit UV lights towards e.g. each shelf 208 arranged within the compartment 204. As discussed above, each of the UV lighting arrangements 104 may e.g. comprise pairs of UV light sources 106/108 emitting light within the first and the second wavelength range, respectively. Alternatively, UV lighting arrangements 104 may comprise UV light sources 106′ emitting light within at least a combine wavelength range extending between at least 250 nm-320 nm.

It may of course be possible and within the scope of the present disclosure to arrange specific UV lighting arrangements 104 within specific compartments (not explicitly shown in FIG. 2A).

In line with the present disclosure, it may be possible to adapt the processing circuitry 112 such that the system 100/100′ emits “enough” UV light for minimizing minimize microorganisms within the compartment 204. In one embodiment, the activation of the system 100/100′ is made dependent on when and for how long the door 206 has been opened. As an alternative or also, the system 100/100′ may be specifically activated once the processing circuitry 112 has received an indication that the compartment 204 has been “filled” with new food, for example following a grocery shopping or once leftover food has been placed in the fridge 202.

In an alternative embodiment, with further reference to FIG. 2B, the system 100/100′ is comprised with a Heating, ventilation, and air conditioning (HVAC) unit 220. The HVAC unit 220 as shows in FIG. 2B is highly simplified.

As is shown, the system 100/100′ is arranged in an air duct 222 of the HVAC unit 220, where the HVAC unit 220 further comprises a filter 224, such as a HEPA filter.

As air passes through the air duct 222 and past the UV light emitted by the plurality of UV lighting arrangements 104, whereby the UV light destroys bacteria, yeasts, mould spores, viruses and other biological contaminants on the surfaces of the air duct 222 and the filter 224.

Further UV lighting arrangements 104 may be included, for regularly emitting light towards the filter 224. It may also be possible to include sensors means for controlling when to operate the system 100/100′, for example if the sensor means determine that contaminants within the passing air is above a predetermined threshold.

It may further be possible to arrange further UV lighting arrangements 104 to emit light towards e.g. HVAC coils (not shown) comprised with the HVAC unit 220. UV light emitted from the UV lighting arrangements 104 to emit light towards the HVAC coils may be used for disinfecting and eliminating or reducing mold from the HVAC coils, which improves the quality of the indoor air, and keeps the HVAC coil consistently clean, which in some embodiment may save significant energy during use.

Additionally, it may be possible to arrange UV lighting arrangements 104 according to the present disclosure in relation to a drain pan (not shown) comprised with the HVAC unit 220. Also here the UV light emitted by the UV lighting arrangements 104 may be used for eliminating or reducing mold, mildew and other bio-growth.

In a further non-shown embodiment, the system 100/100′ may be arranged in a disinfection cabinet. The UV lighting arrangements 104 are here arranged in a manner corresponding to FIGS. 1A and 1B, such that the UV light from the UV lighting arrangements 104 is emitted towards the object to be disinfected within the disinfection cabinet.

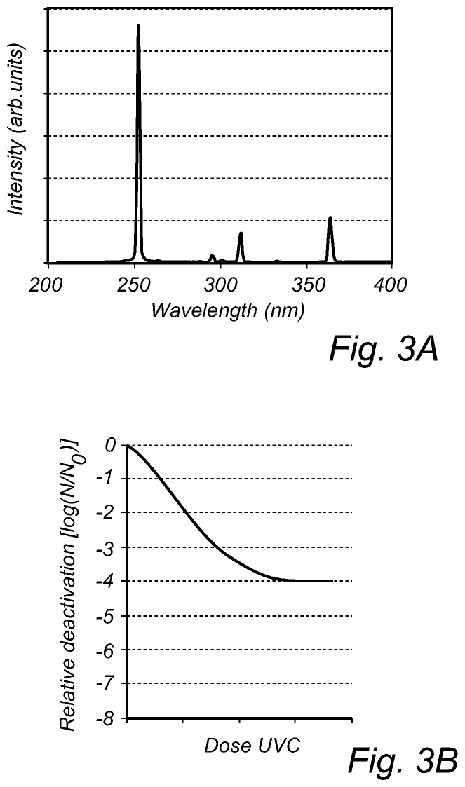

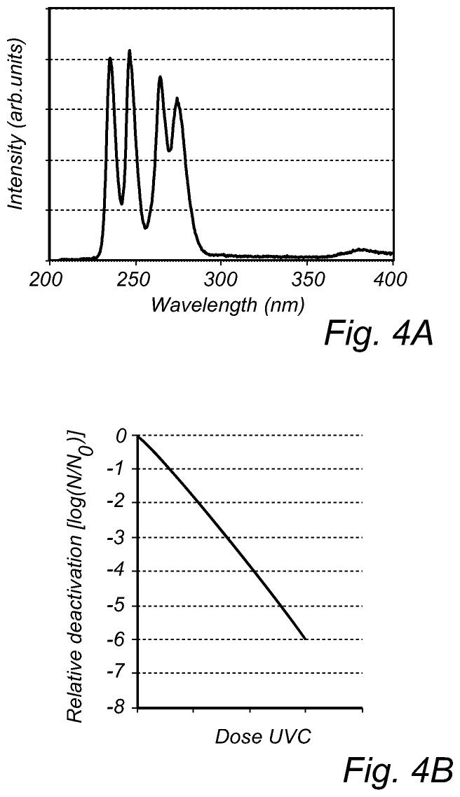

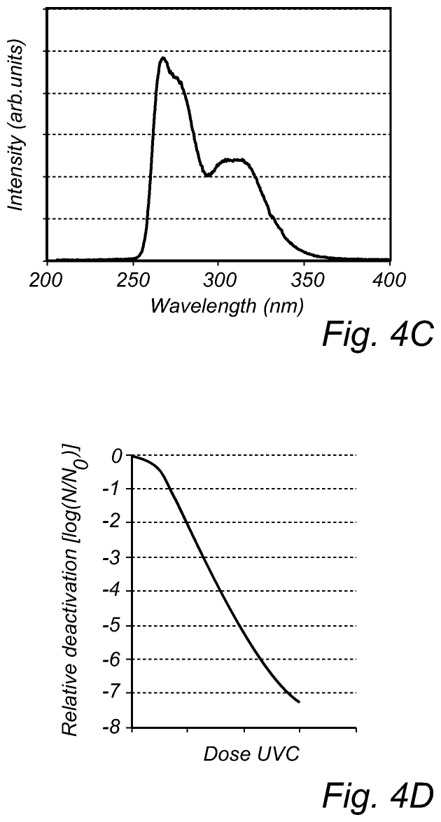

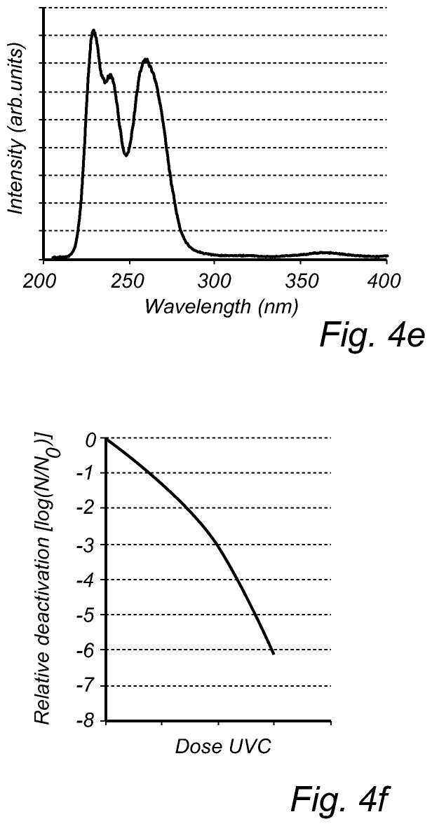

Turning now to FIGS. 3A and 3B, 4A-4F. Note that all measured de-activation curves show the relative reduction as function of UV dose in order to be comparable, thus the vertical axis shows the logarithm of the ratio between the remaining concentration of E. coli in Colony Forming Units per milliliter (CFU/ml)—denoted N— the initial concentration before irradiation, denoted No, thus denoted log(N/No).

As can be seen in FIG. 3A, a LP-Hg lamp essentially emits a strong relatively sharp peak at around 254 nm. FIG. 3B shows the corresponding deactivation of Escherichia coli (E. coli) at a surface. As can be seen, a certain level of E. coli is reached after which no further reduction is seen, i.e. the curve flattens over time at a set level.

Turning now to FIGS. 4A-4F, providing examples of results of use of the exemplary disinfection system shown in FIGS. 1A and 1B for de-activation of E. coli, where UV light is emitted within a wavelength range extending between at least 250 nm-310 nm. Note that all measured de-activation curves show the relative reduction as function of UV dose in order to be comparable, thus the vertical axis shows the logarithm of the ratio between the remaining concentration of E. coli in Colony Forming Units per milliliter (CFU/ml)—denoted N— the initial concentration before irradiation, denoted No, thus denoted log(N/No).

In FIG. 4A, the emission spectra from an UVC field emission light source provided with a first phosphor material (light powder) for UV light emission is provided. In FIG. 4A, the phosphor material has been selected to be a LuPO₄:Pr⁴⁺ phosphor material (or equivalent). In FIG. 4B, the corresponding de-activation curve is shown, for disinfection of water, where no significant tailing is visible.

In FIG. 4C, a second phosphor material in the form of a Lu₂Si₂O₇:Pr⁴⁺ phosphor material is used, and FIG. 4D shows the corresponding de-activation curve. As may be seen, in FIG. 4D, a de-activation of almost 8 orders of magnitude has been achieved, i.e. 99.999999% of the bacteria have been de-activated.

Turning to FIGS. 4E and 4F, where a third phosphor material in the form of a LaPO₄:Pr⁴⁺ phosphor material is used and the corresponding de-activation curve is shown, respectively. The further disclosed electron-excitable UV-emitting material YBO₄:Pr⁴⁺ and YPO₄:Bi⁴⁺ provides similar results as shown in FIGS. 4A-4F.

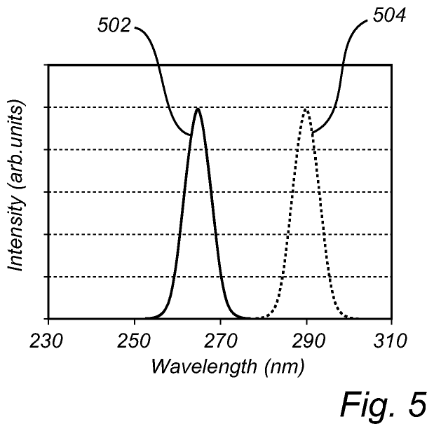

Turning finally to FIG. 5, which illustrates emission spectrums for possible implementation of the system. Specifically, FIG. 5 illustrates an embodiment of the surface treating system according to the present disclosure, where the UV lighting arrangements 104 has been arranged to include two separate UV light sources. Specifically, in FIG. 5 two different UV LEDs have been included with the UV lighting arrangements 104 and their respective emission spectrums 502, 504 are exemplified. Magnitudes of the spectrums are normalized to be equal. In this implementation a first UV LED is selected to have an emission peak a centered at 265 nm (where the germicidal effect may be the largest) and a second UV LED having an emission peak at 290 nm in order to prevent re-activation. It should be understood that the magnitudes of the light sources must not necessarily be exactly equal, and that the emission peaks/center wavelengths may be chosen in other ways depending on the implementation at hand.

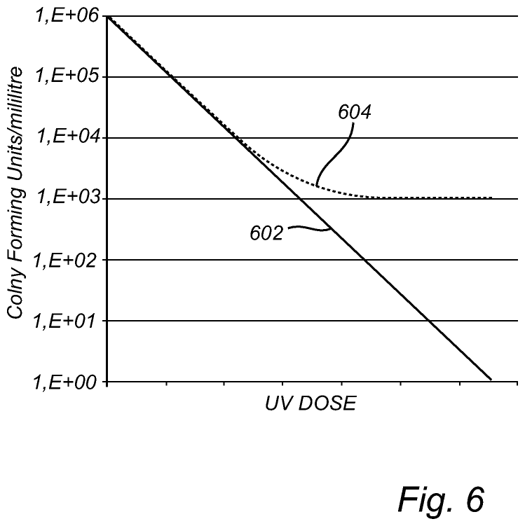

In FIG. 6, a mathematical model is used to describe the effect of inhibiting re-activation (and subsequent regrowth). In principle, the deactivation probability and the re-activation probability are set fixed. In this example the re-activation probability is 0.001 times the de-activation probability, an arbitrarily set number, but which gives results close to measured data. It is further postulated that the UV light can only de-activate non-de-activated organisms, and that the re-activation can only take place in de-activated organisms. As can be seen from FIG. 6 this will give a steady state situation, and a tailing effect will occur.

Looking back at the achieved test results (shown in FIGS. 3B and 4B) it is evident that the behavior is well explained by this model. There may very well be an second effect, such as a portion of microorganisms being more resistant to UV and requiring a higher dose of UV, but from these tests this second effect is at least not dominating, in fact must be much smaller than the re-activation effect. In FIG. 6 the reference 602 illustrates a mathematical modelling of the de-activation behavior with and a re-activation process and the reference 604 illustrates the mathematical modelling of the de-activation behavior without the re-activation process.

In summary, the present disclosure relates to a system for treating a surface, comprising a UV lighting arrangement configured to emit UV light towards the surface, processing circuitry configured to control the operation of the UV lighting arrangement, wherein the UV lighting arrangement under the control of the processing circuitry is adapted to emit UV light within both a first and a second wavelength range to effectively reduce microorganisms at the surface, the first wavelength range has an upper limit extending up to at least 270 nm, and the second wavelength range has a lower limit extending down to at least 270 nm and an upper limit range extending to at least 320 nm. As elaborated above, the emission within these ranges does not need to cover the ranges fully.

In accordance to the present disclosure, the failing effect may also be reduced in relation to surfaces, such as for example in relation to surfaces that may come in direct or indirect contact with a person. An advantage following with the present disclosure is thus that the risk of a decease involving the human/person.

Although the figures may show a specific order of method steps, the order of the steps may differ from what is depicted. In addition, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. Additionally, even though the present disclosure has been described with reference to specific exemplifying embodiments thereof, many different alterations, modifications and the like will become apparent for those skilled in the art.

Variations to the disclosed embodiments can be understood and effected by the skilled addressee in practicing the claimed present disclosure, from a study of the drawings, the disclosure, and the appended claims. Furthermore, in the claims, the word “comprising” does not exclude other elements or steps, and the indefinite article “a” or “an” does not exclude a plurality.

1. A system for treating a surface, comprising:
a UV lighting arrangement configured to emit UV light towards the surface;
processing circuitry configured to control the operation of the UV lighting arrangement,
wherein:
the UV lighting arrangement under the control of the processing circuitry is adapted to emit UV light within both a first and a second wavelength range to effectively reduce microorganisms at the surface,
the first wavelength range has an upper limit extending up to at least 270 nm, and
the second wavelength range has a lower limit extending down to at least 270 nm and an upper limit range extending to at least 320 nm.

2. The system according to claim 1, wherein the first wavelength range extends at least down to at least 250 nm.

3. The system according to claim 1, wherein the UV lighting arrangement comprises a first UV light source adapted to emit UV light within the first wavelength range and a second UV light source adapted to emit UV light within the second wavelength range.

4. The system according to claim 3, wherein the first UV light source is a low-Pressure HG-lamp.

5. The system according to claim 3, wherein the second UV light source is a UVC Light Emitting Diode (LED).

6. The system according to claim 3, wherein at least one of the first and the second UV light source comprises a UV LED.

7. The system according to claim 1, wherein the UV lighting arrangement comprises a non-mercury based UV light source adapted to emit UV light within both the first and the second wavelength range.

8. The system according to claim 7, wherein:
the first wavelength range is between 250-270 nm,
the second wavelength range is between 270-320 nm, and
the non-mercury based UV light source is configured to emit UV light within all of the first and the second wavelength range.

9. The system according to claim 7, wherein the non-mercury based UV light source is a field emission based UV light source.

10. The system according to claim 9, wherein the field emission-based UV light source comprises a light converting material arranged to receive electrons and to emit UV light.

11. The system according to claim 10, wherein the light converting material is selected to be at least one of LaPO4:Pr³⁺, LuPO3:Pr³⁺, Lu2Si2O7:Pr³⁺, YBO3:Pr³⁺ or YPO4:Bi³⁺ or a similar light converting material.

12. The system according to claim 10, wherein the light converting material is a phosphor material.

13. The system according to claim 7, wherein the processing circuitry is adapted to operate of the non-mercury based UV light source according to a predefined schedule.

14. The system according to claim 13, wherein the predefined schedule is dependent on at least one of a distance to the surface, a target micro-organism, or an expected user behavior.

15. The system according to claim 1, wherein the UV lighting arrangement comprises a plurality of UV light sources.

16. A refrigerator, comprising a system according to claim 1, wherein the UV lighting arrangement is arranged to emit the UV light towards an inside surface of the refrigerator.

17. An air purifier, comprising a filter and a system according to claim 1, wherein the UV lighting arrangement is arranged to emit the UV light towards a surface of the filter.

18. The air purifier according to claim 17, wherein the air purifier is comprised with a HVAC unit.

19. A HVAC unit comprising a system according to claim 1, wherein the UV lighting arrangement is arranged to emit the UV light towards an inside surface of the HVAC unit or an inside surface of a component of the HVAC unit.

20. A disinfection cabinet comprising a system according to claim 1.
